(19) United States
(12) Plant Patent Application Publication
(10) Pub. No.: US 2015/0033424 P1
(43) Pub. Date: Jan. 29, 2015

(54) Beautyberry plant named 'NCCX1'

(50) ;

(71) Applicant: North Carolina State University, Raleigh, NC (US)

(72) Inventor: Thomas G. Ranney, Raleigh, NC (US)

(73) Assignee: North Carolina State University, Raleigh, NC (US)

(21) Appl. No.: 13/987,427

(22) Filed: Jul. 24, 2013

(51) Int. Cl. A01H 5/00

(52) U.S. Cl. PLT/226

(57) ABSTRACT

‘NCCX1’ is a new beautyberry plant particularly distinguished by an upright rounded habit, pink/purple flower color (RHS 75A), purple young shoots and emerging leaves, long peduncles and abundant purple fruit.

### LATIN NAME OF THE GENUS AND SPECIES

The Latin name of the novel plant variety disclosed herein is Callicarpa sp.

### VARIETY DENOMINATION

The inventive variety of Callicarpa sp. disclosed herein has been given the varietal denomination ‘NCCX1’.

### BACKGROUND OF THE INVENTION

The present invention comprises a new and distinct variety of Callicarpa, botanically known as Callicarpa sp. and hereinafter referred to by the variety name ‘NCCX1’. ‘NCCX1’ was developed and selected in Mills River, N.C. ‘NCCX1’ is an open-pollinated seedling from Callicarpa kwangtungensis, NCSU 2005-249, (unpatented) planted adjacent to Callicarpa dicotoma, NCSU 2004-024, (unpatented) and appears to be an interspecific hybrid between those two species based on intermediate morphological characters. The first asexual propagation of ‘NCCX1’ was carried out in June 2008 by rooting stem cuttings in Mills River, N.C. and has been asexually reproduced repeatedly by vegetative cuttings in North Carolina over a four year period.

Callicarpa sp. ‘NCCX1’ roots readily from softwood cuttings treated with a basal dip of 4,000 ppm indole butyric acid (potassium salt) in water. ‘NCCX1’ has been found to retain its distinctive characteristics through successive asexual propagations via vegetative cuttings.

### SUMMARY OF THE INVENTION

The following are the unique and distinguishing characteristics of this new cultivar when grown under normal horticultural practices in Mills River, N.C.

1. Pink/purple flowers with an average of 40 flowers per inflorescence.

2. Purple young shoots and emerging leaves.

3. Abundant purple fruit with long peduncles.

### BRIEF DESCRIPTION OF THE DRAWINGS

This new beautyberry variety is illustrated by the accompanying photographs which show the plant's form, foliage, flowers and fruit. The colors shown are as true as can be reasonably obtained by conventional photographic procedures. Colors in the photographs may differ slightly from the color values cited in the detailed botanical description, which accurately describe the colors of the new Callicarpa sp. variety. The photographs were taken in Mills River, N.C.

FIG. 1 is a color photograph of fruit on a mature plant taken on Oct. 26, 2010.

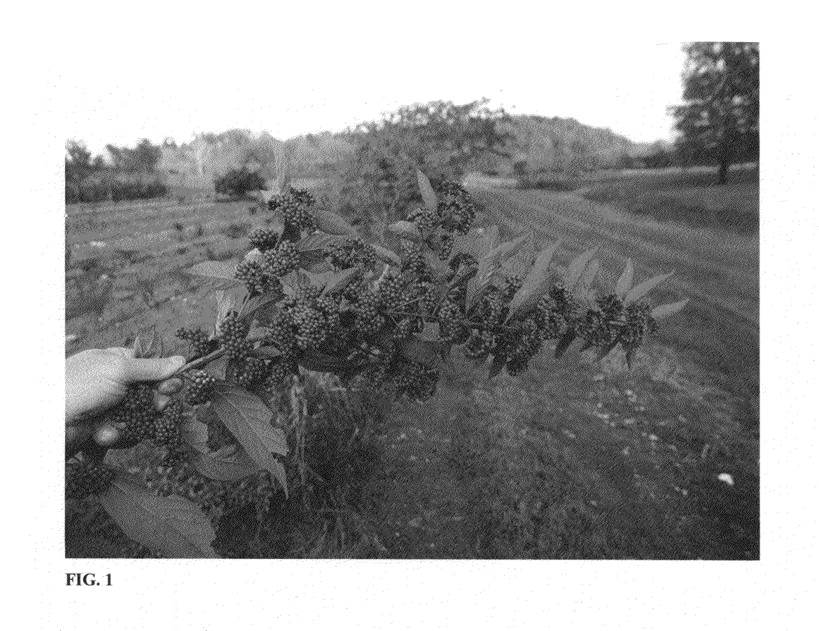

FIG. 2 is a color photograph of the flowers, taken on Sept. 29, 2009.

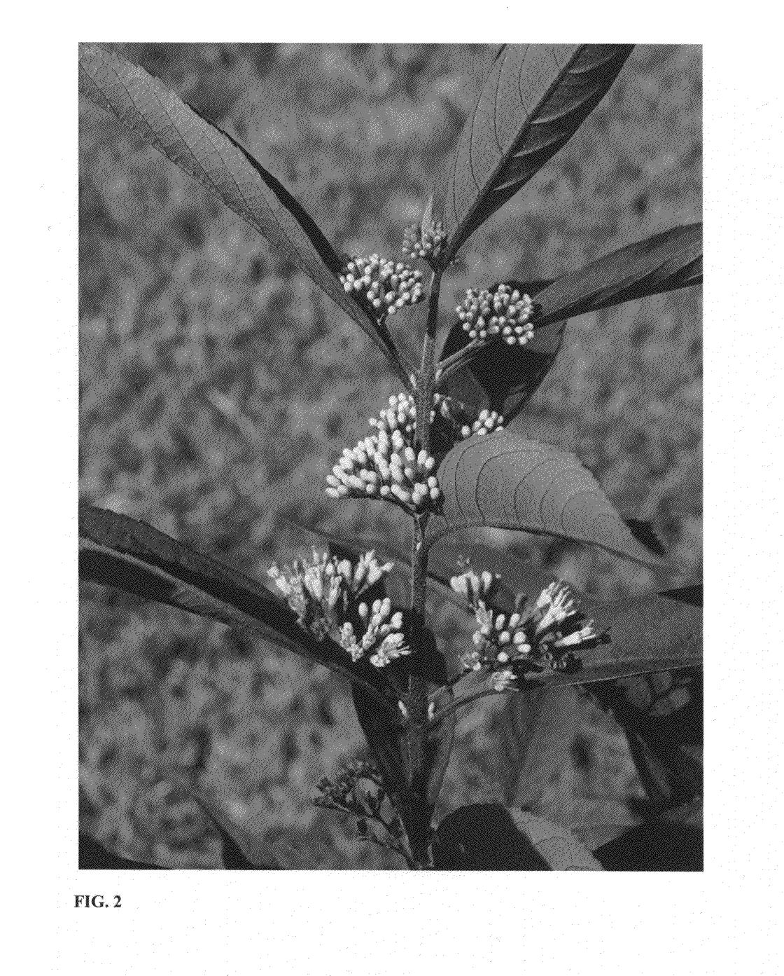

### DETAILED BOTANICAL DESCRIPTION

The following is a detailed description of the botanical characteristics of the new and distinct variety of Callicarpa sp. plant known by the denomination ‘NCCX1’. The detailed description was taken on five-year-old plants growing under landscape conditions in Mills River, N.C. in June 2012. All colors cited herein refer to The Royal Horticultural Society Colour Chart (The Royal Horticultural Society (R.H.S.), London, 2001 Edition). Where specific dimensions, sizes, colors, and other characteristics are given, it is to be understood that such characteristics are approximations or averages set forth as accurately as practicable.
Technical description of the variety:
Classification:
   Botanical name.—Callicarpa sp.
   Common name.—beautyberry.
   Denomination.—‘NCCX1’.
Parentage: Open-pollinated seedling from Callicarpa kwangtungensis (NCSU 2005-249).
Plant description:
   Growth habit.—Upright rounded.
   Plant shape.—Rounded.
   Height at maturity.—2 meters to 2.5 meters.
   Width at maturity.—2 meters to 2.5 meters.
Shoot:
   Mature shoot texture.—Slightly pubescent.
   Mature shoot color.—Closest to RHS 201A (Grey).
   Immature shoot texture.—Pubescent with stellate hairs, especially near tip.
   Immature shoot color.—RHS N77A (Purple).
Leaves:
   Type.—Simple.
   Arrangement.—Opposite.
   Shape.—Elliptic to ovate-lanceolate.
   Apex.—Long acuminate.
   Base.—Cuneate.
   Veins.—Type: Pinnate. Adaxial & abaxial vein color: Closest to RHS 59B (Red-Purple) Surface texture: Pubescent.
   Margin.—Serrate above middle.
   Mature leaf size.—Length: Average is 9.8 cm (range is 6.7 cm to 10.1 cm) Width at widest: Average is 2.9 cm (range is 2.6 cm to 3.1 cm).
   Immature leaf color.—Adaxial (upper) surface: Closest to RHS N77A (Purple) Abaxial (lower) surface: Closest to RHS 148A (Yellow-Green).
   Immature Leaf texture.—Adaxial surface texture: Pubescent Abaxial surface texture: Pubescent.
   Mature leaf color.—Adaxial (upper) surface: Closest to RHS 147A (Yellow-Green) Abaxial (lower) surface: RHS 147B (Yellow-Green) Texture: Adaxial: Sparsely pubescent with glandular hairs Abaxial: Sparsely pubescent with glandular hairs Fall color: Variable, with shades of RHS 186A (Greyed Purple), RHS 22A (Yellow-Orange), and RHS 168A (Greyed-Orange) Leaf attachment: Petiolate Petiole: Shape: Semi-terete Length: Average is 0.7 cm (range is 0.3 cm to 0.8 cm) Width: Average is 0.155 cm (range is 0.105 cm to 0.207 cm) Surface: Sparsely pubescent.
Inflorescence:
   Inflorescence type.—Cyme.
   Inflorescence diameter.—Average is 3.4 cm (range is 2 cm to 4.6 cm).
   Inflorescence length.—Average is 3.4 cm (range is 2.2 cm to 4.2 cm).
   Number of flowers per inflorescence.—Average is 40 (range is 11 to 88).
   Flowering season.—Beginning in June and lasting until October in Mills River, N.C.
   Lastingness of flower on the plant.—Begins June 14 till late Fall or Early Winter.
   Fragrance.—No significant fragrance observed.
   Self cleaning or persistent.—Self cleaning.
   Bud.—Bud shape: Oblong-oval Bud diameter at balloon stage: Average 0.146 cm (range is 0.142 cm to 0.154 cm) Bud length at balloon stage: Average 0.447 cm (range is 0.427 cm to 0.47 cm) Bud color: RHS 75C (Purple).
   Perianth.—Diameter at antithesis: Average 0.425 cm (range is 0.306 cm to 0.552 cm).
   Petal.—Number of petals per flower: 4 Color, adaxial (upper) surface at anthesis: RHS 75A (Purple) Color, abaxial (lower) surfaces at anthesis: Ranging between RHS 75A (Purple) and RHS 75B (Purple) Surface texture and appearance: Glabrous Shape: Campanulate flower form Apex: Acute Base: Petals fused together at base Margin: Entire above fused region Length: Average is 0.461 cm (range is 0.454 cm to 0.484 cm) Width: Average is 0.155 cm (range is 0.132 cm to 0.177 cm) Petals, fused or unfused: Fused.
   Peduncle.—Color: RHS N77A (Purple) Length: Average 1.6 cm (range is 1 cm to 2.6 cm) Width: Average 0.087 cm (range is 0.066 cm to 0.106 cm) Shape: Cylindrical to semi-terete Surface texture: Pubescent with stellate hairs.
   Bract.—Number: 2 to 12 (or more) per inflorescence Length: Average 0.2 cm (range is 0.1 cm to 0.7 cm) Width: Average 0.052 cm (range is 0.033 cm to 0.111 cm) Color: RHS N77A (Purple).
   Sepals.—Number: 4 Color: RHS N77A (Purple) Surface texture: Sparsely glandular Shape: Funnel form Apex: Abruptly acute Base: Fused Margin: Entire above fused region Length: Average 0.242 cm (range is 0.210 cm to 0.264 cm) Width: Average 0.111 cm (range is 0.10 cm to 0.123 cm).
Reproductive organs:
   Stamen.—Quantity per flower: 4 Shape: Oblong elliptic, exerted beyond petals Filament color at anthesis: Translucent RHS 155A (White) Length: Average is 0.496 cm (range is 0.471 cm to 0.534 cm) Width of filament: Average 0.0069 cm (range is 0.0048 cm to 0.010 cm).
   Anther.—Length: Average is 0.183 cm (range is 0.167 cm to 0.205 cm) Width at widest: Average is 0.0724 cm (range is 0.032 cm to 0.102 cm) Dehiscence: Poricidal; dehiscing through a pore-like opening at the apex Color: RHS 9C (Yellow) Pollen color: RHS 9C (Yellow) Attachment: dorsifixed.
   Pistil.—Length: Average 0.742 cm (range 0.682 cm to 0.806 cm) Style Length: Average 0.6 cm (range 0.529 cm to 0.64 cm) Style Width at midpoint: Average 0.023 cm (range is 0.015 cm to 0.029 cm) Ovary: Superior.
Fruit/seed set:
   Fruit.—Color: RHS N78A (Purple) Shape: globose Surface texture: glabrous, shiny, glossy Size: Average is 0.498 cm (range is 0.439 cm to 0.521 cm) Amount of fruit set: very abundant.
   Seed.—Color: RHS 18B (Yellow-orange) Shape: ovoid, flattened Length: Average is 0.115 cm (range is 0.08 cm to 0.135 cm) Width: Average is 0.059 cm (range is 0.047 to 0.08 cm) Surface texture: Glabrous.
Disease and insect resistance: No significant disease or insect pests have been observed.
Comparison with parental lines and known varieties: Callicarpa sp. ‘NCCX1’ is distinguished from the female parent Callicarpa kwantungensis NCSU 2005-249 (unpatented) in that ‘NCCX1’ has an upright rounded habit, pink/purple flower color (RHS 75A), more flowers per inflorescence (average of 40), longer peduncles (average length of 1.6 cm) and more abundant, larger (average diameter of 0.5 cm), purple (RHS N78A) fruit, while C. kwantungensis has a narrow upright habit, white flowers (RHS 155B), shorter peduncles (average length of 0.25 cm), and smaller (average diameter of 0.37 cm), less abundant, purple-violet (RHS N80A) fruit.

Table 1 shows the specific comparisons of Callicarpa sp. ‘NCCX1’ to Callicarpa bodinieri var. giraldii ‘Profusion’ NCSU 2001-064, Callicarpa kwantungensis NCSU 2005-249, and Callicarpa dichotoma ‘Issai’ NCSU 2006-204.

TABLE 1
Comparison of Callicarpa sp. ‘NCCX1’ with other Callicarpa taxa.

| | | | | |
|---|---|---|---|---|
| Habit | full, rounded and | upright and open | narrow upright; | dense upright |
| | upright | | stiff straight stems | |
| Shoot color | dark purple new | dark gray (RHS | gray-brown (RHS | dark purple new |
| | shoots (RHS N77A) | 197A); no | 199A); no | shoots (RHS N77A) |
| | | significant purple | significant purple | |
| | | color on new | color on new | |
| | | shoots | shoots | |
| Leaf color | emerging leaves | both emerging and | emerging leaves | both emerging and |
| | purple (RHS N77A); | mature leaves are | purple (RHS | mature leaves green |
| | expanded leaves | medium green | N79A-N77A); | (RHS 146B) |
| | green (RHS 147A) | (RHS 144A) to | mature leaves | |
| | | deep green (RHS | overall dull olive | |
| | | 147A) | green (RHS 152A) | |
| Leaf | purple mid-rib (RHS | purple mid-rib | purple mid-rib | purple mid-rib (RHS |
| venation | 59B) extends about | (RHS N77A) | (RHS N77A) | N77A) extends |
| | 46% of length from | extends about 12% | extends 100% from | about 8% from |
| | lamina base to tip | of length from | lamina base to tip | lamina base to tip |
| | | lamina base to tip | | |
| Petiole | dark purple (RHS | dark purple (RHS | dark purple (RHS | dark purple (RHS |
| | N77A); average | N77A); average | N77A); average | N77A); average |
| | length 0.7 cm (range | length 1.48 cm | length 1.15 cm | length 0.49 cm |
| | 0.3 to 0.8 cm) | (range 0.8 to 2.1 cm) | (range 0.8 to 1.5 cm) | (range 0.3 to 0.6 cm) |
| Flowering | mid June | late May | late June | mid June |
| time in Mills | | | | |
| River, NC | | | | |
| Flowers per | Average 40 | Average 17 | Average 17 | Average 15 |
| inflorescence | (range is 11 to 88) | (range is 4 to 32) | (range is 12 to 27) | (range is 12 to 25) |
| Flower color | pink/purple | medium purple- | white (RHS 155B) | pale purple-pink |
| | (RHS 75A) | pink (RHS N78A) | | (closest to RHS 75A) |
| Flower | on long peduncles; | short peduncles; | short peduncles | held proud from |
| position | not held in ranks | cymes well- | clustered and | foliage in ranks |
| | | branched with fruit | hidden in leaf axil | along shoot |
| | | encircling shoot | | |
| Peduncle | Average 1.6 cm | Average 0.548 cm | Average 0.252 cm | Average 0.591 cm |
| length | (range is 1 to 2.6 cm) | (range is 0.369 to | (range is 0.201 to | (range is 0.455 to |
| | | 0.962 cm) | 0.315 cm) | 0.72 cm) |
| Fruit | very abundant | abundant | minimal | very abundant |
| abundance | | | | |
| Fruit color | vibrant purple (RHS | red-purple (RHS | purple-violet (RHS | vibrant purple |
| | N78A) | 72A) | N80A) | (RHS N81A) |
| Fruit size | 0.5 cm average per | 0.48 cm average | 0.37 cm average | 0.34 cm average per |
| | drupe | per drupe | per | drupe |
| | | | drupe | |
| Maturity of | beginning to ripen in | beginning to ripen | beginning to ripen | beginning to ripen |
| fruit | late August, peak | in August, peak | in September, peak | in August, peak fruit |
| | fruit color in late | fruit color in | fruit color in late | color in mid |
| | September to frost | September to frost | September to frost | September to frost |

What is claimed is:

1. A new and distinct variety of beautyberry plant named ‘NCCX1,’ substantially as illustrated and described herein.
